(19) United States
(12) Patent Application Publication
(10) Pub. No.: US 2024/0066015 A1
(43) Pub. Date: Feb. 29, 2024

(54) TREATMENT OF PAIN ASSOCIATED WITH PARKINSON'S DISEASE

(71) Applicant: BIAL-PORTELA & CA., S.A., S. Mamede de Coronado (PT)

(72) Inventors: Patrício Soares Da Silva, S. Mamede de Coronado (PT); José Francisco Da Costa De Pinho Rocha, S. Mamede de Coronado (PT)

(21) Appl. No.: 18/273,515

(22) PCT Filed: Aug. 4, 2021
(86) PCT No.: PCT/PT2021/050026

§ 371 Date: Jul. 20, 2023

(30) Foreign Application Priority Data
Jan. 20, 2021 (WO) PCT/PT2021/050001

(51) Int. Cl. A61K 31/4439; A61K 31/165; A61K 31/198; A61P 25/16

(52) U.S. Cl. CPC A61K 31/4439; A61K 31/165; A61K 31/198; A61P 25/16

(57) ABSTRACT

This invention relates to methods for treating pain associated with Parkinson's disease (PD). In particular it relates to the use of opicapone for treating PD-associated pain, especially fluctuation-related pain.

### FIELD OF THE INVENTION

This invention relates to methods for treating pain associated with Parkinson's disease (PD). In particular it relates to the use of opicapone for treating PD-associated pain, especially fluctuation-related pain.

### BACKGROUND OF THE INVENTION

Although motor complications are the most well-known symptoms of PD, James Parkinson himself noted and described non-motor symptoms (NMS) of the disease which would come to bear his name. These symptoms included sleep problems, bowel dysfunction and pain. However, research into, and treatment of, PD has historically focused on controlling motor symptoms, and it is only relatively recently that the importance of NMS has become widely recognised [Martinez-Fernandez et al, 2016].

Pain is one of the most frequent and burdensome NMS of PD, being a significant comorbidity in up to 85% of PD patients, and it may precede the motor symptoms of the disease. Pain has been shown to be associated with other NMS of PD such as sleep disruption and cardiovascular disturbances, indicating that pain, sleep disruption and dysautonomia may share a common pathophysiology that possibly involves non-dopaminergic pathways [Ghosh et al, 2020]. On the other hand, nociceptive pain accounts for the majority of reported pain in PD [Truini et al, 2013]. Dopamine can modulate pain and is known to have an anti-nociceptive role [Allen et al, 2015], and dopaminergic therapies have been shown to help alleviate pain in PD [Brefel-Courbon et al, 2005, 2013; Antonini et al, 2018; Rukavina et al, 2019. It is therefore possible that pain in PD may have, at least partially, a dopamine-associated aetiology [Antonini et al, 2018; Seppi et al, 2019]. Consequently, optimisation of dopaminergic therapy may help alleviate pain associated with PD [Jung et al, 2015; Antonini et al, 2018; Rukavina et al, 2019; Dafsari et al, 2019]. However, clinical evidence for the benefit of dopaminergic therapies in PD-associated pain is lacking [Seppi et al 2019], and previous studies in this setting have notable limitations.

The Phase II PANDA trial was the first randomised controlled trial to specifically assess treatment for PD-associated pain. Eligible patients were randomised to receive either prolonged-release oxycodone-naloxone or placebo. There was no significant difference between treatment arms in the average 24-h pain score at 16 weeks (primary endpoint). However, the measure used to assess pain was a general pain scale (the Likert scale) and levodopa was used more frequently as a rescue treatment in the placebo arm, both of which factors might have affected the results [Trenkwalder et al, 2015].

The double-blind, exploratory DOLORES trial was the first to investigate the effect of a dopamine agonist (rotigotine; administered as a transdermal patch) on PD-associated pain as primary outcome. Although the findings suggested that rotigotine may improve PD-associated chronic pain in patients with advanced-stage PD, the trial was not powered to detect statistically significant treatment differences, due to the small sample size [Rascol et al, 2016].

Safinamide (an agent with multiple modes of action, including monoamine oxidase-B inhibition) was shown to significantly reduce the need for pain medication, and to significantly improve two out of three PDQ-39 pain-related items, in comparison with placebo, when added to existing levodopa-based therapy [Cattaneo et al, 2017]. The authors noted that safinamide is a state-dependent inhibitor of human voltage-gated sodium channels (VGSC) in the inactivated state and that it might regulate selective inhibition of glutamatergic hyperactivity, which may be an effective strategy for the treatment of some PD non-motor symptoms not responding to levodopa therapy, in particular pain and neuropsychiatric symptoms. Therefore, any effect might be independent of safinamide's effect on monoamine oxidase-B.

Levodopa is still the most effective symptomatic treatment for PD [Poewe et al, 2010]. However, following oral administration, levodopa is extensively metabolised in the periphery by dopa decarboxylase (DDC) and catechol-O-methyltransferase (COMT), with the result that only 1% of an oral dose of levodopa reaches the brain. Moreover, long-term treatment with levodopa is complicated by the development of “wearing off” periods (characterised by end-of-dose motor fluctuations) and drug-induced dyskinesia [Poewe et al, 2010]. PD associated pain often increases during such OFF periods and patients with dyskinesia often have increased pain sensitivity [Cheon et al, 2009; Sung et al, 2020].

Inhibitors of DDC (DDCIs) and COMT (COMTIs) are commonly used as an adjunct to levodopa in patients with PD in order to increase levodopa bioavailability and its delivery to the brain, and thereby ameliorate wearing-off symptoms, but potentially exasperating dyskinesia [Müller, 2015; Montioli et al, 2016]. According to WO01/68083, COMTIs may be used to treat or control pain of any origin, including acute and chronic pain. However, the evidence supporting this claim is limited to the use of nitecapone and entacapone in two animal models of inflammatory pain (i.e. a modified Randall-Sellitto test and the acetic acid induced writhing test). A third experiment in an animal model of centrally mediated analgesia (the hot plate test) indicated that the analgesic effect of nitecapone and entacapone was not mediated through the CNS, but through some (unidentified) peripheral mechanism. The skilled person is aware that different sub-types of pain respond to different treatments and an effect on acute pain in animal models cannot indicate an effect in alternative pain paradigms.

Opicapone is a third-generation, once-daily COMTI [Kiss et al, 2010; Almeida et al, 2013; Scott, 2016; Fabbri et al 2018], which has been shown to be generally well tolerated and efficacious in reducing OFF-time in two pivotal studies in patients with PD and end-of-dose motor fluctuations (BIPARK-I and BIPARK-II) [Ferreira et al, 2016; Lees et al, 2017]. On the basis of these trials, opicapone is approved in the European Union, USA, Japan, Australia and other countries as adjunctive therapy to preparations of levodopa/DDCIs in patients with PD and end-of-dose motor fluctuations [Ongentys ° EU Summary of Product Characteristics] or OFF episodes [Ongentys® USA Prescribing Information]. Although a positive signal for opicapone was observed on the Movement Disorder Society Non-Motor Symptoms Scale (NMSS) “Miscellaneous” domain, which includes pain as one aspect, in both the BIPARK-II trial [Oliveira et al, 2015] and the more recent OPTIPARK study [Reichmann et al, 2020], the effect was less than for that observed for placebo (in the BIPARK-II trial), and no specific effect on pain was identifiable. Placebo is known to activate dopamine receptors and to induce dopamine-like effects in PD [de la Fuente-Fernandez et al, 2002; Benedetti, 2014; Colloca, 2019; Lou, 2020].

### SUMMARY OF THE INVENTION

As a result of their detailed knowledge of the properties and effects of opicapone and their understanding of the problem of pain associated with PD, the present inventors elected to investigate the efficacy of opicapone in PD patients with end-of-dose motor fluctuations and associated pain, when administered as adjunctive therapy to existing treatment with levodopa/DDCI. Such insight has resulted in conception of the present invention as summarised below.

A first general embodiment relates to opicapone (or a pharmaceutically acceptable derivative thereof) in combination with levodopa (or a pharmaceutically acceptable derivative thereof) for use in the treatment of pain associated with Parkinson's disease.

A second general embodiment relates to the use of opicapone (or a pharmaceutically acceptable derivative thereof) in the manufacture of a medicament for use, in combination with levodopa (or a pharmaceutically acceptable derivative thereof) in the treatment of pain associated with Parkinson's disease.

A third general embodiment relates to a method of treating pain associated with Parkinson's disease comprising administering a therapeutically effect amount of opicapone (or a pharmaceutically acceptable derivative thereof) in combination with a therapeutically effect amount of levodopa (or a pharmaceutically acceptable derivative thereof) to a patient identified as suffering from pain associated with Parkinson's disease.

### BRIEF DESCRIPTION OF THE DRAWINGS

Examples of the invention will be described in detail with reference to the accompanying drawings, in which:

FIG. 1 shows the study design of a randomised, double-blind, placebo-controlled, parallel-group, interventional trial in PD patients with end-of-dose motor fluctuations and PD-associated pain. It is noted that V2 is divided in V2a and V2b. If ON/OFF diary entries are non-compliant at V2a, the patient will be re-trained on correct use of the diary and visit V2b will be postponed for 3-4 days. If diary completion is satisfactory at V2a, V2b is performed immediately on the same day. In FIG. 1, AE=adverse event; DDCI=dopa decarboxylase inhibitor; L-dopa=levodopa; PD=Parkinson's disease; PSV=post-study visit; V=visit.

FIG. 2 shows the timelines of study assessments. In FIG. 2, CGI-C=Clinical Global Impression of Change; DDCI=dopa decarboxylase inhibitor; EMD=early morning dystonia; KPPS=King's Parkinson's Disease Pain Scale; L-dopa=levodopa; MDS-NMS=Movement Disorder Society-sponsored Non-Motor rating Scale; MDS-UPDRS=Movement Disorder Society-sponsored Unified Parkinson's Disease Rating Scale; PDQ-8=8-item Parkinson's Disease Questionnaire; PGI-C=Patient's Global Impression of Change; PSV=post-study visit; V=visit.

### DETAILED DESCRIPTION OF THE EMBODIMENTS

In a first embodiment, the present invention provides opicapone (or a pharmaceutically acceptable derivative thereof) in combination with levodopa (or a pharmaceutically acceptable derivative thereof) for use in the treatment of pain associated with Parkinson's disease.

In a second embodiment, the present invention provides the use of opicapone (or a pharmaceutically acceptable derivative thereof) in the manufacture of a medicament for use, in combination with levodopa (or a pharmaceutically acceptable derivative thereof) in the treatment of pain associated with Parkinson's disease.

In a third embodiment, the present invention provides a method of treating pain associated with Parkinson's disease comprising administering a therapeutically effect amount of opicapone (or a pharmaceutically acceptable derivative thereof) in combination with a therapeutically effect amount of levodopa (or a pharmaceutically acceptable derivative thereof) to a patient identified as suffering from pain associated with Parkinson's disease. Thus, the method of the third embodiment involves the initial step of identifying patients who not only suffer from Parkinson's disease, but also pain associated therewith, and who will therefore benefit from the present invention.

For each of the three embodiments described above, the following paragraphs set out preferred aspects of the invention.

### Primary Indication

The primary goal of the present invention is the treatment of pain associated with Parkinson's disease. The severity of pain associated with Parkinson's disease may be measured using the King's Parkinson's Disease Pain Scale (KPPS). The KPPS evaluates the burden and characterises various phenotypes of pain in PD. It comprises seven domains including a total of 14 items. The domains are:
1. Musculoskeletal pain (pain around the joints (including arthritic pain));
2. Chronic pain (generalized or organ specific);
3. Fluctuation-related pain (dyskinetic pain, “OFF” dystonia or generalised “OFF” period pain);
4. Nocturnal pain (pain related to jerking leg movements or an unpleasant burning sensation in the legs which improves with movement, or pain related to difficulty turning in bed);
5. Orofacial pain (pain when chewing, pain due to teeth grinding at night, or burning mouth syndrome);
6. Discoloration, edema/swelling (burning pain in limbs (often associated with swelling or dopaminergic treatment) or generalised lower abdominal pain); and
7. Radicular pain (shooting pain/pins and needles down the limbs).

Each item is scored by severity (0, none to 3, very severe) multiplied by frequency (0, never to 4, all the time), resulting in subscores of 0-12. The total KPPS score (with a theoretical range of 0-168) represents the symptomatic burden by pain [Chaudhuri et al, 2015].

Treatment according to the present invention results in a reduction in a patient's score in one or more of these seven domains. A particular population who will benefit from the present invention are patients having a total score greater than or equal to 40 in the KPPS. Preferably, treatment according to the present invention reduces the patient's total score in the KPPS, preferably to a score less than or equal to 8, more preferably to a score less than or equal to 10, most preferably to a score less than or equal to 12.

Whilst pain related to Parkinson's disease is not necessarily related to end-of-dose motor fluctuations and can occur at all stages of disease, it is preferable that the patient to be treated experiences end-of-dose motor fluctuations. End-of-dose motor fluctuations (also known as the “wearing off” phenomenon) are a well-known consequence of extended levodopa therapy. They relate to the predictable re-emergence or worsening of symptoms before administration of the next dose of levodopa. Typically, such re-emergence or worsening of symptoms starts 3 to 4 hours after the last dose of levodopa, as the therapeutic effect of the medication wears off. Symptoms then typically improve 15 to 45 minutes after the next levodopa dose is taken.

In patients experiencing end-of-dose motor fluctuations, the pain associated with Parkinson's disease is preferably pain associated with end-of-dose motor fluctuations, such as dyskinetic pain, “OFF” dystonia or generalised “OFF” period pain. The severity of this type of pain may be measured as fluctuation-related pain according to domain 3 of the KPPS. A particular population who will benefit from the present invention are patients having a score greater than or equal to 12 in domain 3 of the KPPS. Preferably, treatment according to the present invention reduces the patient's score in domain 3 of the KPPS, preferably to a score less than or equal to 2, more preferably to a score less than or equal to 3, most preferably to a score less than or equal to 4. The reduction may involve a reduction in one or more of the sub-domains—dyskinetic pain, “OFF” dystonia or generalised “OFF” period pain.

Alternatively, or additionally, the pain associated with Parkinson's disease is nocturnal pain. The severity of this type of pain may be measured as nocturnal pain according to domain 4 of the KPPS. A particular population who will benefit from the present invention are patients having a score greater than or equal to 8 in domain 4 of the KPPS. Preferably, treatment according to the present invention reduces the patient's score in domain 4 of the KPPS, preferably to a score less than or equal to 0.5, more preferably to a score less than or equal to 1, most preferably to a score less than or equal to 2.

### Patient Group

The patient is suffering from pain associated with Parkinson's disease, preferably the patient to be treated also experiences end-of-dose motor fluctuations. Such patients to be treated according to the invention may have been experiencing a mean daily OFF time of up to 8 hours per day prior to treatment according to the invention, preferably 0.5 to 8 hours, 1 to 8 hours, 2 to 8 hours, 4 to 8 hours, 5 to 8 hours per day prior to treatment according to the invention. Patients experiencing end-of-dose motor fluctuations tend to have been treated for longer periods, for example, more than 1 year, preferably more than 2 years, more preferably more than 3 years, even more preferably more than 4 years, most preferably more than 5 years prior to treatment according to the invention. The patients to be treated according to the invention may have been experiencing motor symptoms and/or motor complications such as motor fluctuations with or without dyskinesias prior to treatment according to the invention, for example more than 1 month, preferably more than 3 months, more preferably more than 6 months, even more preferably more than 1 year, most preferably more than 2 years prior to treatment according to the invention.

A preferred patient group includes those who have only been experiencing end-of-dose motor fluctuations for a relatively short period of time. Such patients to be treated according to the invention may have been experiencing a mean daily OFF time of up to 4 hours per day prior to treatment according to the invention, preferably up to 3 hours, more preferably up to 2 hours, even more preferably up to 1 hour, most preferably up to 0.5 hours, per day prior to treatment according to the invention. Patients who have only been experiencing end-of-dose motor fluctuations for a relatively short period of time tend to have been treated for shorter periods, for example, less than 5 years, preferably less than 4 years, more preferably less than 3 years, even more preferably more less than 2 years, most preferably less than 1 year prior to treatment according to the invention. The patients to be treated according to the invention may have been experiencing motor symptoms and/or motor complications such as motor fluctuations with or without dyskinesias prior to treatment according to the invention, for example less than 2 years, preferably less than 1 year, more preferably less than 6 months, even more preferably less than 3 months, most preferably less than 1 month prior to treatment according to the invention.

### Secondary Indications

In addition to suffering from pain associated with Parkinson's disease, the patient to be treated may also experience one or more further non-motor symptoms and, preferably, the treatment reduces one or more of these symptoms too. The severity of non-motor symptoms may be assessed using the Movement Disorder Society Non-Motor Scale (MDS-NMS). The MDS-NMS comprises 13 domains covering a range of key PD- and treatment-related non-motor symptoms, and a subscale for non-motor fluctuations that assesses changes in non-motor symptoms in relation to timing of anti-PD medications across eight domains [Martinez-Martin et al, 2019; Chaudhuri et al, 2020]. Preferably, treatment according to the present invention reduces the patient's total score in the MDS-NMS.

A particular non-motor symptom which may be experienced in addition to pain associated with Parkinson's disease (in particular during “OFF” periods) is anxiety and, preferably, the treatment reduces this symptom too. Preferably, the treatment reduces the patient's score in domain B of the MDS-NMS.

Another non-motor symptom which may be experienced in addition to pain associated with Parkinson's disease (in particular during “OFF” periods) is depression and, preferably, the treatment reduces this symptom too. Preferably, the treatment reduces the patient's score in domain A of the MDS-NMS.

Another non-motor symptom which may be experienced in addition to pain associated with Parkinson's disease is sleep disorders and, preferably, the treatment reduces this symptom too. Preferably, the treatment reduces the patient's score in domain K of the MDS-NMS.

### Treatment Regimens

In the context of the present invention, the phrase “pharmaceutically acceptable derivative” of an active ingredient means a compound which is non-toxic and is converted to the active ingredient itself upon administration to the patient. It includes pharmaceutically acceptable salts, solvates and prodrugs (e.g. esters).

In the context of the present invention, wherein the active ingredient opicapone (or a pharmaceutically acceptable derivative thereof) is administered in combination with the active ingredient levodopa (or a pharmaceutically acceptable derivative thereof), the phrase “in combination with” does not mean the two active ingredients must be administered in the same dosage unit. Indeed, they need not even be given at the same point in time. Thus, the phrase “in combination with” simply means that the two active ingredients must exert their pharmacological effects on the patient at the same time. Thus, the two active ingredients may be administered concomitantly, separately or sequentially, in the same or different dosage unit(s), and with the same or different dosing frequency.

The particular dose amount and frequency for opicapone (or a pharmaceutically acceptable derivative thereof) may be determined by the skilled physician. Preferably, it is administered once daily. Preferably each dose is equivalent to 10 to 100 mg of opicapone, more preferably equivalent to 25 to 50 mg of opicapone and most preferably equivalent to 50 mg of opicapone. The most preferred dose amount and frequency is 50 mg, once daily. Opicapone can interact with food and with levodopa. Therefore, it is preferably administered at least 1 hour before or after a meal, and at least 1 hour before or after levodopa. A particularly suitable dosing point is at or near to bedtime, e.g. up to 1 hour before sleep.

The particular dose amount and frequency for levodopa (or a pharmaceutically acceptable derivative thereof) may be determined by the skilled physician. Preferably, it is administered three to ten times daily. Preferably, each dose is equivalent to 50 to 200 mg of levodopa, more preferably equivalent to 75 to 125 mg of levodopa and most preferably equivalent to 100 mg of levodopa. Preferably, the total daily dose is equivalent to 300 to 2000 mg of levodopa, more preferably equivalent to 500 to 1000 mg of levodopa.

Levodopa therapy often benefits from the use of a DOPA decarboxylase inhibitor (DDCI). Therefore, treatment according to the present invention preferably involves administration of a DDCI, preferably selected from carbidopa or benserazide. The particular dose amount and frequency for the DDCI may be determined by the skilled physician. Preferably, it is administered three to ten times daily. Preferably, the total daily dose is equivalent to 25 to 500 mg of the DDCI, more preferably equivalent to 75 to 250 mg of the DDCI.

### Example

A clinical study to exemplify the present invention is described below.

### Study Design

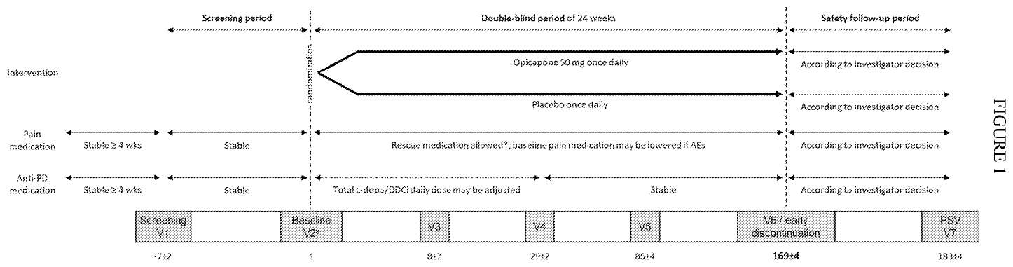

A phase IV, randomised, double-blind, placebo-controlled, parallel-group, interventional trial in PD patients with end-of-dose motor fluctuations and PD-associated pain (experienced for ≥4 weeks prior to the start of the study, with a score of ≥12 on Domain 3 of the KPPS at screening and baseline). It consists of a 1-week screening period, 24-week double-blind treatment period and 2-week follow-up period (FIG. 1). Following screening, at visit (V)2, eligible patients will be randomised 1:1 to opicapone 50 mg or placebo (PLC) once daily while continuing current treatment with levodopa/DDCI. Since opicapone enhances the effects of levodopa, it may be necessary to reduce the patient's levodopa/DDCI dosing within the first days or weeks of opicapone treatment; therefore, the investigator may decrease the daily dose of levodopa/DDCI as required until V4, while keeping the number of daily intakes unchanged. If necessary, dosing may be increased back to the baseline dose level. After V4, the levodopa/DDCI dose should not be changed until the end of the study. The anti-PD treatment regimen should be stable for at least 4 weeks prior to V1 (Table 1) and kept stable throughout the study (except for levodopa/DDCI during the adjustment period). No new anti-PD drugs should be started during the study.

Chronic pain treatment should be stable for at least 4 weeks prior to V1 (Table 1), and no new pain medication should be started during the study, except the allowed rescue medication (paracetamol or tramadol). The baseline dose of pain medication may be reduced throughout the study, if required due to pain medication-related adverse events (AEs), and increased again up to the baseline dose level if the investigator finds that the dose reduction was too much. Further visits will be performed on Day 85±4 days (V5) and Day 169±4 days (V6). The primary analysis will be performed on data collected at V6. A follow-up visit will be performed on Day 183±4 days (V7), approximately 2 weeks after the last intake of study medication (opicapone 50 mg or PLC). Patients who discontinue early will be requested to attend an early discontinuation visit. At V6 (or early discontinuation visit, if applicable), the investigator will arrange the patient's subsequent treatment (i.e. either prescribe further opicapone or switch to another treatment).

### Study Population

Inclusion and exclusion criteria are outlined in Table 1 below.

### Study Assessments

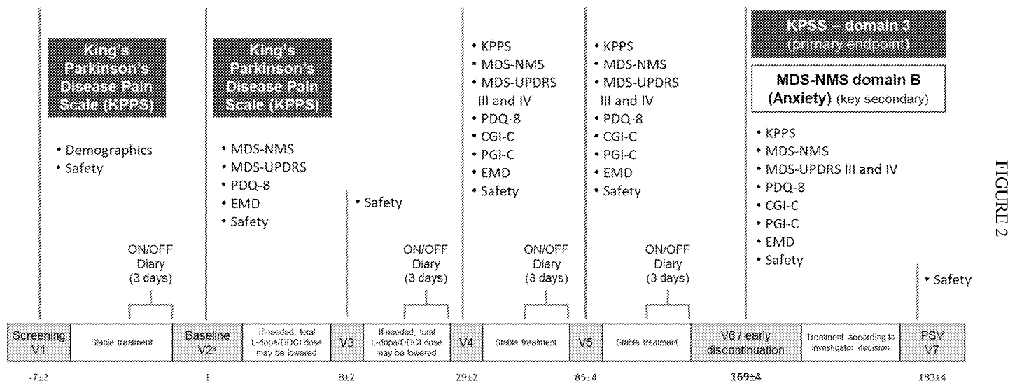

An overview of study assessments is presented in Table 2 below and the timing of these assessments is outlined in FIG. 2.

### Efficacy

The primary efficacy endpoint is change from baseline in Domain 3 (fluctuation-related pain) of the KPPS. The KPPS evaluates the burden and characterises various phenotypes of pain in PD. It comprises seven domains including a total of 14 items. Each item is scored by severity (0-3) multiplied by frequency (0-4), resulting in subscores of 0-12. The total KPPS score (0-168) represents the symptomatic burden by pain [Chaudhuri et al, 2015].

The key secondary efficacy endpoint is change from baseline in Domain B (anxiety) of the Movement Disorder Society-sponsored Non-Motor rating Scale (MDS-NMS). The MDS-NMS comprises 13 domains covering a range of key PD- and treatment-related non-motor symptoms, and a subscale for non-motor fluctuations that assesses changes in non-motor symptoms in relation to timing of anti-PD medications across eight domains [Martinez-Martin et al, 2019; Chaudhuri et al, 2020].

Additional secondary efficacy endpoints comprise other domains and total scores of KPPS and MDS-NMS, change from baseline in Movement Disorder Society-sponsored Unified Parkinson's Disease Rating Scale (MDS-UPDRS) Parts III and IV, change from baseline in Parkinson's Disease Questionnaire (PDQ-8), Clinical Global Impression of Change (CGIC), Patient's Global Impression of Change (PGIC), change from baseline in functional status via Hauser's PD diary, changes from baseline in morning dystonia, and use of rescue medication (see Table 2 below).

The MDS-UPDRS is a revision of the UPDRS originally developed in the 1980s, and evaluates various aspects of PD; it consists of four parts: Parts IA and IB, non-motor aspects of experiences of daily living; Part II, motor aspects of experiences of daily living; Part III, motor examination; and Part IV, motor complications. The PDQ-8 (a short form of the PDQ-39) is a patient-reported outcome that assesses eight aspects of functioning and well-being that are usually adversely affected by PD: mobility, activities of daily living, emotional well-being, stigma, social support, cognition, communication, and bodily discomfort. It rates overall health status by providing a single score ranging from 0 (good health) to 100 (poor health). The CGIC and PGIC are, respectively, investigator and patient assessments of how much a patient's overall status has improved or worsened since the start of the study, comprising a 7-point scale: (1, ‘very much improved’; 2, ‘much improved’; 3, ‘minimally improved’; 4, ‘no change’; 5, ‘minimally worse’; 6, ‘much worse’; 7, ‘very much worse’). The Hauser's PD diary is a patient record of their mobility during each 30-min period, categorised as: asleep; OFF time; ON time without dyskinesia; ON time with non-troublesome dyskinesia; or ON time with troublesome dyskinesia. When assessing changes from baseline in morning dystonia, the investigator will ask the patient if they experienced any morning dystonia within the last week (based on item 35 of the former UPDRS version). The amount and frequency of intake of rescue medication (paracetamol or tramadol) will be recorded by patients in a diary.

### Safety Assessments

Safety assessments include the incidence of treatment-emergent adverse events (TEAEs), and changes from baseline in vital signs, physical and neurological examinations and routine laboratory parameters (see Table 2; and FIG. 2).

### Sample Size Calculation

For the primary efficacy endpoint (change from baseline in Domain 3 of KPPS), a difference to PLC of 3.0 is regarded as clinically meaningful. From a former study [Rascol et al, 2016], a standard deviation (SD) of 5.8 can be assumed. With a two-sided significance a of 0.05, a power of 80%, a 1:1 treatment allocation ratio and with the above-mentioned assumptions, 2×60=120 evaluable patients are required. Assuming a drop-out rate of 15%, a total of 140 patients need to be randomised. Randomisation will follow a 1:1 allocation rate (opicapone 50 mg or PLC).

### Statistical Methodology

Efficacy assessments will be analysed for the Full Analysis Set, defined as all patients who are randomised and who have at least one measurement of the primary efficacy assessment. For sensitivity purposes, efficacy assessments will additionally be analysed for the Per-Protocol Set, defined as all patients included in the Full Analysis Set who have no major protocol deviations that could influence the primary efficacy assessment. The primary efficacy endpoint will be analysed using analysis of covariance (ANCOVA), with treatment as a fixed factor and baseline KPPS as a covariate, to demonstrate superiority of opicapone 50 mg against PLC. Secondary efficacy endpoints will be analysed in an exploratory manner by treatment arm using appropriate parametric and non-parametric statistical methods. Descriptive statistics, including 95% confidence intervals, will be presented per treatment arm.

Safety assessments will be analysed for the Safety Set, defined as all patients who take at least one dose of investigational product. TEAEs will be summarised in terms of the number and percentages of patients with TEAEs. Vital signs and laboratory parameters will be summarised using summary statistics of absolute values and changes from baseline. Summary statistics and shift tables will be presented for physical and neurological examinations. Demographic and baseline characteristics will be presented using descriptive statistics.

### Discussion

The robust design of the above study addresses the current lack of reliable evidence for levodopa-based therapy in the treatment of PD-associated pain. The study features recent validated PD pain- and non-motor-specific scales (such as KPPS and MDS-NMS), which help to record dimensions of PD-associated pain that were not previously possible to assess. For instance, this may allow the detection of potential associations between pain and other non-motor symptoms, such as depression, anxiety and insomnia, and dysautonomic symptoms. The concomitant use of ON/OFF diaries with these scales may also allow a deeper understanding of pain during both the OFF and ON states. Placebo is known to activate dopamine receptors and to induce dopamine-like effects in PD [de la Fuente-Fernandez et al, 2002; Benedetti, 2014; Colloca, 2019; Lou, 2020], which are often still apparent in studies at 3 months [Trenkwalder et al, 2015, Ferreira et al, 2016; Lees et al, 2017], tending to wane by the 6-month mark [Borgohain et al, 2014; Hattori et al, 2020]. The 6 months course of the study and its double-blind design may therefore help to disentangle the placebo effect from the true effect, especially when evaluating pain.

TABLE 1
Inclusion and exclusion criteria

| Category of | | |
|---|---|---|
| characteristic | Inclusion criteria | Exclusion criteria |
| Demographics | Male or female | |
| | Age ≥30 years^{a} | |
| Disease-related | Disease severity Stages I-III | Non-idiopathic PD^{c} |
| characteristics | at ON^{b} | Severe and/or unpredictable |
| | Signs of ‘wearing-off’ | OFF periods (investigator's |
| | phenomena (end-of-dose | judgment) |
| | fluctuations) with average | |
| | total daily OFF time while | |
| | awake ≥1.5 h (excluding | |
| | early morning pre-first dose | |
| | OFF period), despite optimal | |
| | anti-PD therapy, according | |
| | to the investigator's | |
| | judgment at V1 | |
| | At least 1.5 OFF h/day | |
| | (excluding early morning | |
| | pre-first dose OFF period), | |
| | as recorded in the self-rated | |
| | diary, during at least 2 of the | |
| | 3 days prior to V2 | |
| Pain-related | Experiencing PD-associated | Major/prominent non-PD- |
| characteristics | pain for ≥4 weeks prior to | related pain (e.g. due to |
| | V1 | malignant disease) |
| | Domain 3 of KPPS ≥12 at | |
| | V1 and V2 | |
| | No changes in chronic | |
| | treatment regimen for pain | |
| | within 4 weeks prior to V1^{d} | |
| Anti-PD | Treated with 3-8 intakes/day | Treatment with prohibited |
| medication | of levodopa/DDCI^{e} and on a | medication^{f} within the 4 weeks |
| | stable regimen for ≥4 weeks | prior to V1 |
| | prior to V1 | Treatment with apomorphine |
| | Any other anti-PD | with 4 weeks prior to V1 or |
| | medication regimen, if | likely to be needed at any time |
| | applicable, should remain | until V6 |
| | stable for ≥4 weeks prior to | Previous or planned (during |
| | V1 and should not be likely | the entire study duration) |
| | to require any adjustment | levodopa/carbidopa intestinal |
| | until V6 | gel infusion, deep brain |
| | | stimulation or stereotactic |
| | | surgery (e.g. pallidotomy, |
| | | thalamotomy) |
| | | Previous or current use of |
| | | opicapone |
| | | Use of any other |
| | | investigational product, |
| | | currently or within 3 months |
| | | (or five half-lives of the |
| | | investigational product, |
| | | whichever is longer) prior to |
| | | V1 |
| Compliance | Adequate compliance with | |
| | relevant PD and pain-related | |
| | medication during the | |
| | screening period | |
| | (investigator's judgment) at | |
| | V2 | |
| | Filled in the self-rating diary | |
| | in accordance with the diary | |
| | instructions and with ≤3 | |
| | missing entries/day in the 3 | |
| | days prior to V2 | |
| Safety | Acceptable results of | Current or past (within |
| | screening laboratory tests | previous year) history of |
| | (i.e. not clinically relevant | suicidal ideation, suicide |
| | for the well-being of the | attempts or alcohol or |
| | patient or for the purpose of | substance abuse, excluding |
| | the study according to | caffeine or nicotine |
| | investigator's judgment at | Pheochromocytoma, |
| | V2 | paraganglioma or other |
| | For female patients: | catecholamine-secreting |
| | postmenopausal for ≥2 years | neoplasms |
| | before V1, surgically sterile | Known hypersensitivity to the |
| | for ≥6 months before V1, or | excipients of the investigation |
| | practicing effective | product^{h} or rescue medication |
| | contraception until V6^{g} | History of neuroleptic |
| | For male patients: use of | malignant syndrome or non- |
| | condoms plus an approved | traumatic rhabdomyolysis |
| | method of highly effective | History of severe hepatic |
| | contraception during the | impairmentⁱ |
| | treatment period up to V6, if | Previous history of psychosis |
| | sexually active with a | or psychiatric disorders, |
| | partner of childbearing | including severe major |
| | potential | depression |
| | | Any medical condition that |
| | | might place the patient at |
| | | increased risk or interfere with |
| | | assessments |
| | | For female patients: pregnant |
| | | or breastfeeding |

^{a}According to UK Parkinson's Disease Society Brain Bank Clinical Diagnostic Criteria (2006) or Movement Disorder Society Clinical Diagnostic Criteria for Parkinson's disease (2015).
^{b}Modified Hoehn and Yahn staging.
^{c}Atypical parkinsonism, secondary (acquired or symptomatic) parkinsonism, Parkinson-plus syndrome.
^{d}Includes medication (e.g. paracetamol, opioids, nonsteroidal anti-inflammatory drugs, antidepressants, anticonvulsants, corticosteroids) and non-medication therapies (e.g. transcutaneous electrical nerve stimulation, bioelectrical therapy).
^{e}May include a slow-release formulation.
^{f}Entacapone, tolcapone, monoamine oxidase inhibitors (except selegiline up to 10 mg/day [oral] or 1.25 mg/day [buccal], rasagiline up to 1 mg/day, safinamide up to 100 mg/day) or antiemetics with anti-dopaminergic action (except domperidone).
^{g}Female patients requesting to continue with oral contraceptives must be willing to additionally use non-hormonal methods of contraception during the course of the study.
^{h}Including lactose intolerance, galactose intolerance, Lapp lactase deficiency or glucose-galactose malabsorption.
ⁱChild-Pugh Class C.
DDCI, dopa decarboxylase inhibitor; KPPS, King's Parkinson's Disease, Pain Scale; PD, Parkinson's disease; V, visit.

TABLE 2
Overview of study assessments

| Category | Assessment |
|---|---|
| Primary efficacy | Change from baseline in Domain 3 (fluctuation-related pain) of |
| endpoint | KPPS |
| Key secondary | Change from baseline in Domain B (anxiety) of MDS-NMS |
| endpoint | |
| Additional secondary | Change from baseline in Domain A (depression) of MDS-NMS |
| endpoints | Change from baseline in Domain K (sleep and wakefulness) of |
| | MDS-NMS |
| | Change from baseline in MDS-NMS total score |
| | Change from baseline in Domain 4 (nocturnal pain) of KPPS |
| | Change from baseline in KPPS total score |
| | Change from baseline in MDS-UPDRS Parts III and IV |
| | Change from baseline in PDQ-8 |
| | CGIC |
| | PGIC |
| | Change from baseline in functional status via Hauser's PD diary |
| | Changes from baseline in morning dystonia |
| | Use of rescue medication^{a} |
| Safety assessments | Incidence of TEAEs, including serious TEAEs |
| | Changes from baseline in vital signs |
| | Changes from baseline in physical and neurological examinations |
| | Changes from baseline in routine laboratory parameters^{b} |

^{a}Paracetamol or tramadol;
^{b}haematology, serum biochemistry, pregnancy test.
CGIC, Clinical Global Impression of Change; KPPS, King's Parkinson's Disease Pain Scale; MDS-NMS, Movement Disorder Society-sponsored Non-Motor rating Scale; MDS-UPDRS, Movement Disorder Society-sponsored Unified Parkinson's Disease Rating Scale; PD, Parkinson's disease; PDQ-8, 8-item Parkinson's Disease Questionnaire; PGIC, Patient's Global Impression of Change; TEAE, treatment-emergent adverse event.

### REFERENCES

Allen et al. The Rationale for Exercise in the Management of Pain in Parkinson's Disease. J Parkinsons Dis. 2015; 5(2):229-39.
Almeida et al. Pharmacokinetics, pharmacodynamics and tolerability of opicapone, a novel catechol-O-methyltransferase inhibitor, in healthy subjects: prediction of slow enzyme-inhibitor complex dissociation of a short-living and very long-acting inhibitor. Clin Pharmacokinet. 2013 February; 52(2):139-51.
Antonini et al. Pain in Parkinson's disease: facts and uncertainties. Eur J Neurol. 2018 July; 25(7):917-e69.
Benedetti F. Placebo effects: from the neurobiological paradigm to translational implications. Neuron. 2014 Nov. 5; 84(3):623-37.
Borgohain et al. Randomized trial of safinamide add-on to levodopa in Parkinson's disease with motor fluctuations. Mov Disord. 2014 February; 29(2):229-37.
Brefel-Courbon et al. Effect of levodopa on pain threshold in Parkinson's disease: a clinical and positron emission tomography study. Mov Disord. 2005 December; 20(12):1557-63.
Brefel-Courbon et al. Nociceptive brain activation in patients with neuropathic pain related to Parkinson's disease. Parkinsonism Relat Disord. 2013 May; 19(5):548-52.
Cattaneo et al. Long-term Efficacy of Safinamide on Parkinson's Disease Chronic Pain. Adv Ther. 2018 April; 35(4):515-522.
Chaudhuri et al. King's Parkinson's disease pain scale, the first scale for pain in PD: An international validation. Mov Disord. 2015 October; 30(12):1623-31.
Chaudhuri et al. The movement disorder society nonmotor rating scale: Initial validation study. Mov Disord. 2020 January; 35(1):116-133.
Cheon et al. Non-motor off symptoms in Parkinson's disease. J Korean Med Sci. 2009 April; 24(2):311-4.
Colloca L. The Placebo Effect in Pain Therapies. Annu Rev Pharmacol Toxicol. 2019 Jan. 6; 59:191-211.
Dafsari et al: Subthalamic stimulation, apomorphine, and levodopa infusion in Parkinson's disease. Mov Disord. 2019 March; 34(3):353-365.
de la Fuente-Fernandez et al. The placebo effect in neurological disorders. Lancet Neurol. 2002 June; 1(2):85-91.
Fabbri et al. Opicapone for the treatment of Parkinson's disease: A review of a new licensed medicine. Mov Disord. 2018 October; 33(10):1528-1539.
Ferreira et al. Opicapone as an adjunct to levodopa in patients with Parkinson's disease and end-of-dose motor fluctuations: a randomised, double-blind, controlled trial. Lancet Neurol. 2016 February; 15(2):154-165.
Ghosh et al. A Dual Centre Study of Pain in Parkinson's Disease and Its Relationship with Other Non-Motor Symptoms. J Parkinsons Dis. 2020; 10(4):1817-1825.
Hattori et al. Efficacy and safety of safinamide as an add-on therapy to L-DOPA for patients with Parkinson's disease: A randomized, double-blind, placebo-controlled, phase II/III study. Parkinsonism Relat Disord. 2020 June; 75:17-23.
Jung et al. An 8-Year Follow-up on the Effect of Subthalamic Nucleus Deep Brain Stimulation on Pain in Parkinson Disease. JAMA Neurol. 2015 May; 72(5):504-10.
Kiss et al. Discovery of a long-acting, peripherally selective inhibitor of catechol-O-methyltransferase. J Med Chem. 2010 Apr. 22; 53(8):3396-411.
Lees et al. Opicapone as Adjunct to Levodopa Therapy in Patients With Parkinson Disease and Motor Fluctuations: A Randomized Clinical Trial. JAMA Neurol. 2017 Feb. 1; 74(2):197-206.
Lou J S. Placebo responses in Parkinson's disease. Int Rev Neurobiol. 2020; 153:187-211.
Martinez-Fernandez et al. The hidden sister of motor fluctuations in Parkinson's disease: A review on non-motor fluctuations. Mov Disord. 2016 August; 31(8):1080-94.
Martinez-Martin et al. Pilot Study of the International Parkinson and Movement Disorder Society-sponsored Non-motor Rating Scale (MDS-NMS). Mov Disord Clin Pract. 2019 Feb. 5; 6(3):227-234.
Montioli et al. Parkinson's Disease: Recent Updates in the Identification of Human Dopa Decarboxylase Inhibitors. Curr Drug Metab. 2016; 17(5):513-8.
Müller T. Catechol-O-methyltransferase inhibitors in Parkinson's disease. Drugs. 2015 February; 75(2):157-74.
Oliveira et al. Evaluation of non-motor symptoms in opicapone treated Parkinson's disease patients: results from a double-blind, randomized, placebo-controlled study and open-label extension. Eur J Neurol 2015; 22(Suppl 1):191 (abstract P1236).
Ongentys® Summary of Product Characteristics. BIAL—Portela & C^{a} S.A; 2020. Available at: https://wwwemaeuropa.eulen/documentslproduct-information/ongentys-epar-product-information_en.pdf Accessed 7 Dec. 2020.
Ongentys® Prescribing Information. Neurocrine Biosciences, Inc.; 2020. Available at: https://www.accessdataRla.gov/drugsatfda docsliabel/2020/212489s0001b1. df Accessed 7 Dec. 2020.
Poewe et al. Levodopa in the treatment of Parkinson's disease: an old drug still going strong. Clin Intery Aging. 2010 Sep. 7; 5:229-38.
Rascol et al. A Randomized Controlled Exploratory Pilot Study to Evaluate the Effect of Rotigotine Transdermal Patch on Parkinson's Disease-Associated Chronic Pain. J Clin Pharmacol. 2016 July; 56(7):852-61.
Reichmann et al. Effectiveness and safety of opicapone in Parkinson's disease patients with motor fluctuations: the OPTIPARK open-label study. Transl Neurodegener. 2020 Mar. 4; 9(1):9.
Rukavina et al. Pain in Parkinson's disease: new concepts in pathogenesis and treatment. Curr Opin Neurol. 2019 August; 32(4):579-588.
Scott L J. Opicapone: A Review in Parkinson's Disease. Drugs. 2016 September; 76(13):1293-1300.
Seppi et al. Update on treatments for nonmotor symptoms of Parkinson's disease—an evidence-based medicine review. Mov Disord. 2019 February; 34(2):180-198.
Sung et al. Pain and dyskinesia in Parkinson's disease may share common pathophysiological mechanisms—An fMRI study. J Neurol Sci. 2020 May 29; 416:116905.
Trenkwalder et al. Prolonged-release oxycodone-naloxone for treatment of severe pain in patients with Parkinson's disease (PANDA): a double-blind, randomised, placebo-controlled trial. Lancet Neurol. 2015 December; 14(12):1161-70.
Truini et al. Parkinson's disease related pain: a review of recent findings. J Neurol. 2013 January; 260(1):330-4.

1. A method for the treatment of pain associated with Parkinson's disease, comprising administering a therapeutically effective amount of opicapone, or a pharmaceutically acceptable derivative thereof, in combination with a therapeutically effective amount of levodopa, or a pharmaceutically acceptable derivative thereof, to a patient identified as suffering from pain associated with Parkinson's disease.

2. The method according to claim 1, wherein the patient has a total score greater than or equal to 40 in the King's Parkinson's Disease Pain Scale (KPPS).

3. The method according to claim 1, wherein the treatment reduces the patient's total score in the KPPS to a score less than or equal to 8.

4. The method according to claim 1, wherein the patient to be treated experiences end-of-dose motor fluctuations.

5. The method according to claim 4, wherein the pain associated with Parkinson's disease is pain associated with end-of-dose motor fluctuations.

6. The method according to claim 5, wherein the pain associated with end-of-dose motor fluctuations is fluctuation-related pain according to domain 3 of the KPPS.

7. The method according to claim 6, wherein the patient to be treated has a score greater than or equal to 12 in domain 3 of the KPPS.

8. The method according to claim 6, wherein the treatment reduces the patient's score in domain 3 of the KPPS to a score less than or equal to 2.

9. The method according to claim 1, wherein the pain associated with Parkinson's disease is nocturnal pain.

10. The method according to claim 9, wherein the nocturnal pain is nocturnal pain according to domain 4 of the KPPS.

11. The method according to claim 10, wherein the patient to be treated has a score greater than or equal to 8 in domain 4 of the KPPS.

12. The method according to claim 10, wherein the treatment reduces the patient's score in domain 4 of the KPPS to a score less than or equal to 0.5.

13. The method according to claim 1, wherein the patient to be treated experiences one or more further non-motor symptoms and the treatment reduces one or more of those symptoms.

14. The method according to claim 13, wherein the treatment reduces the patient's total score in the Movement Disorder Society Non-Motor Scale (MDS-NMS).

15. The method according to claim 13, wherein the patient to be treated experiences anxiety and the treatment reduces the patient's score in domain B of the MDS-NMS.

16. The method according to claim 13, wherein the patient to be treated experiences depression and the treatment reduces the patient's score in domain A of the MDS-NMS.

17. The method according to claim 13, wherein the patient to be treated experiences sleep disorders and the treatment reduces the patient's score in domain K of the MDS-NMS.

18. The method according to claim 1, wherein said combination further comprises a DOPA decarboxylase inhibitor (DDCI).

19. The method according to claim 1, wherein the opicapone, or a pharmaceutically acceptable derivative thereof, is administered once daily at a dose equivalent to 10 to 100 mg of opicapone.

20. (canceled)

21. (canceled)
